(19) United States
(12) Patent Application Publication
(10) Pub. No.: US 2025/0290115 A1
(43) Pub. Date: Sep. 18, 2025

(54) METHOD FOR DETECTING ANTIMICROBIAL AND ANTIVIRAL EFFECT

(71) Applicant: ASUSTEK COMPUTER INC., TAIPEI (TW)

(72) Inventor: Sheng-Yu TSAI, TAIPEI (TW)

(21) Appl. No.: 18/923,988

(22) Filed: Oct. 23, 2024

(30) Foreign Application Priority Data
Mar. 13, 2024 (CN) 202410284612.1

(51) Int. Cl. C12Q 1/18

(52) U.S. Cl. CPC C12Q 1/18

(57) ABSTRACT

The disclosure provides a method for detecting an antimicrobial and antiviral effect of the surface of a material to be tested. The method comprises: providing a plurality of samples to be tested, wherein the samples to be tested are all made of the material to be tested; respectively coating probiotics on the surfaces to be tested and detecting the surfaces to be tested by using an ATP measuring instrument to generate a plurality of original values; after placing the samples to be tested in the constant-temperature environment for a first time, detecting the surfaces to be tested to generate a plurality of first detection values by using the ATP measuring instrument; calculating a plurality of first inhibition values by using the first detection values and the original values; and confirming the antimicrobial and antiviral effect of the surface of the material to be tested according to the first detection values.

### CROSS-REFERENCE TO RELATED APPLICATION

This application claims the priority benefit of Chinese application serial No. 202410284612.1, filed on Mar. 13, 2024. The entirety of the above-mentioned patent application is hereby incorporated by reference herein and made a part of the specification.

### BACKGROUND OF THE INVENTION

### Field of the Invention

The disclosure relates to a detection method and particularly relates to a method for detecting an antimicrobial and antiviral effect.

### Description of the Related Art

A quick confirmation effect of antimicrobial and antiviral materials cannot be obtained by a quick screening method. The efficacy thereof must be confirmed by a third party notarial unit.

### BRIEF SUMMARY OF THE INVENTION

The disclosure provides a method for detecting an antimicrobial and antiviral effect, which is suitable for detecting the antimicrobial and antiviral effect of the material surface of a material to be tested. The method for detecting an antimicrobial and antiviral effect comprises: providing a plurality of samples to be tested, wherein the samples to be tested are all made of the material to be tested and each sample to be tested is provided with a surface to be tested; respectively coating probiotics on the surfaces to be tested and detecting the surfaces to be tested by using an adenosine triphosphate (ATP) measuring instrument to generate a plurality of original values; after placing the samples to be tested in the constant-temperature environment for a first time, detecting the surfaces to be tested to generate a plurality of first detection values by using the adenosine triphosphate measuring instrument; calculating a plurality of first inhibition values by using the first detection values and the original values; and confirming the antimicrobial and antiviral effect of the material surface of the material to be tested according to the first detection values.

The disclosure further provides another r method for detecting an antimicrobial and antiviral effect, which is suitable for detecting the antimicrobial and antiviral effect of the material surface of a material to be tested. The method for detecting an antimicrobial and antiviral effect comprises: providing a plurality of samples to be tested, wherein the samples to be tested are all made of the material to be tested and each sample to be tested is provided with a surface to be tested; dividing the samples to be tested into a first test group and a second test group; respectively coating probiotics on the surfaces to be tested and detecting the surfaces to be tested by using an adenosine triphosphate measuring instrument to generate a plurality of original values; after placing the samples to be tested of the first test group in the constant-temperature environment for a first time, detecting the surfaces to be tested of the first test group to generate a plurality of first detection values by using the adenosine triphosphate measuring instrument and calculating a plurality of corresponding first inhibition values by using the first detection values and the original values; after placing the samples to be tested of the second test group in the constant-temperature environment for a second time, detecting the surfaces to be tested of the second test group to generate a plurality of second detection values by using the adenosine triphosphate measuring instrument and calculating a plurality of corresponding second inhibition values by using the second detection values and the original values; and confirming the antimicrobial and antiviral effect of the material surface of the material to be tested according to the first inhibition values and the second inhibition values.

The method for detecting an antimicrobial and antiviral effect confirms the antimicrobial effect or the antiviral effectiveness of a test substance in a short time by using the adenosine triphosphate measuring instrument matched with a special detection process, and is helpful to reduce the detection cost and time spent.

### BRIEF DESCRIPTION OF THE DRAWINGS

FIG. 1 is a flow diagram of a method for detecting an antimicrobial and antiviral effect provided by a first embodiment of the disclosure;

FIG. 2A is a schematic view of a test component provided by one embodiment of the disclosure;

FIG. 2B is a schematic view of a test environment provided by one embodiment of the disclosure;

FIG. 2C is a schematic view of a test environment provided by another embodiment of the disclosure;

FIG. 3 shows an embodiment of step S150 in FIG. 1;

FIG. 4 shows an embodiment of step S330 in FIG. 3;

FIG. 5 is a flow diagram of a method for detecting an antimicrobial and antiviral effect provided by a second embodiment of the disclosure;

FIG. 6 is a flow diagram of a method for detecting an antimicrobial and antiviral effect provided by a third embodiment of the disclosure;

FIG. 7 shows an embodiment of step S650 in FIG. 6;

FIG. 8 shows an embodiment of step S740 in FIG. 7;

FIG. 9 is a flow diagram of a method for detecting an antimicrobial and antiviral effect provided by a fourth embodiment of the disclosure;

FIG. 10 shows an embodiment of step S960 in FIG. 9; and

FIG. 11 shows another embodiment of step S960 in FIG. 9.

### DETAILED DESCRIPTION OF THE EMBODIMENTS

The specific embodiments of the disclosure are described in detail below with reference to the accompanying drawings. According to the following description and claims, the advantages and features of the disclosure will be clearer. It should be noted that the drawings all adopt very simplified forms and all use imprecise ratios, which are only used for the purpose of conveniently and clearly assisting in describing the embodiments of the disclosure.

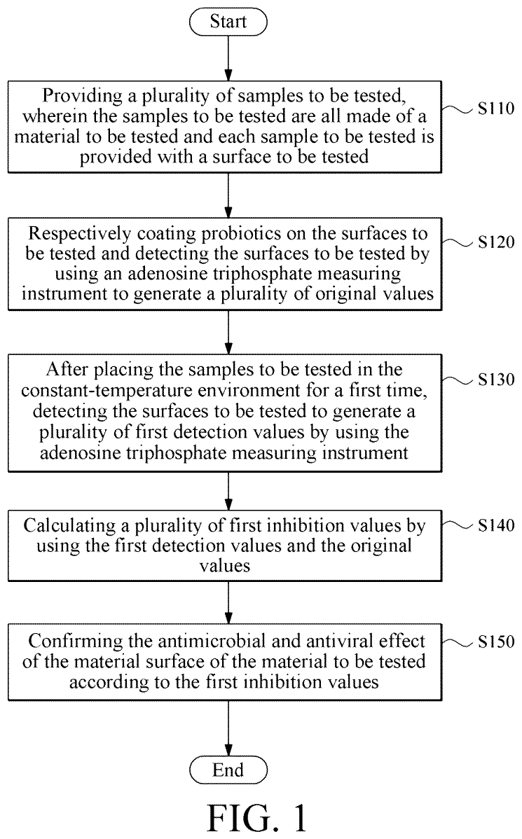

FIG. 1 is a flow diagram of a method for detecting an antimicrobial and antiviral effect provided by a first embodiment of the disclosure. The method for detecting an antimicrobial and antiviral effect is suitable for detecting the antimicrobial and antiviral effect of the material surface of a material to be tested.

The method for detecting an antimicrobial and antiviral effect comprises the following steps.

First, as described in step S110, a plurality of samples to be tested is provided, wherein the samples to be tested are all made of the material to be tested and each sample to be tested is provided with a surface to be tested. In one embodiment, the samples to be tested are cut into square slices. The number of the samples to be tested is three.

Then, as described in step S120, probiotics are respectively coated on the surfaces to be tested and the surfaces to be tested are detected by using an adenosine triphosphate (ATP) measuring instrument to generate a plurality of original values. The adenosine triphosphate measuring instrument utilizes a luciferase to react with adenosine triphosphate (ATP) in microorganisms (probiotics) to emit light, and then the adenosine triphosphate measuring instrument is utilized to calculate the photon number. The reading value presented by the adenosine triphosphate measuring instrument is the photon concentration value and represents the residual amount of the microorganisms. The detection technology is the prior art of biological detection, not essential to the disclosure, and thus not described herein.

In one embodiment, the step of preparing the probiotics into a probiotic solution and coating the probiotic solution on the surfaces to be tested in a titration manner. In one embodiment, the probiotics is Bacillus subtilis. In one embodiment, the original values are relative light units (RLU) obtained by detecting the surfaces to be tested with the adenosine triphosphate measuring instrument.

Then, as described in step S130, after the samples to be tested are placed in the constant-temperature environment for a first time, the surfaces to be tested are detected to generate a plurality of first detection values by using the adenosine triphosphate measuring instrument. In one embodiment, the first detection values are relative light units obtained by detecting the surfaces to be tested with the adenosine triphosphate measuring instrument.

In one embodiment, the sample to be tested is prepared into a test component to be placed in the constant-temperature environment continuously for the first time. The preparation details of the test component are described in subsequent paragraphs. In addition, in one embodiment, the first time is one hour. In one embodiment, the sample to be tested is placed in an incubator as the constant-temperature environment.

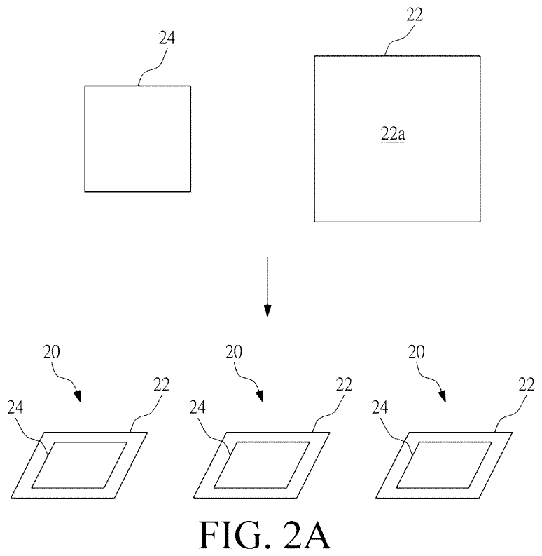

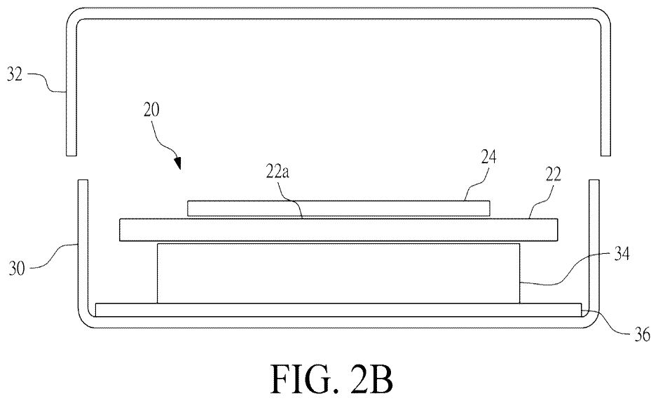

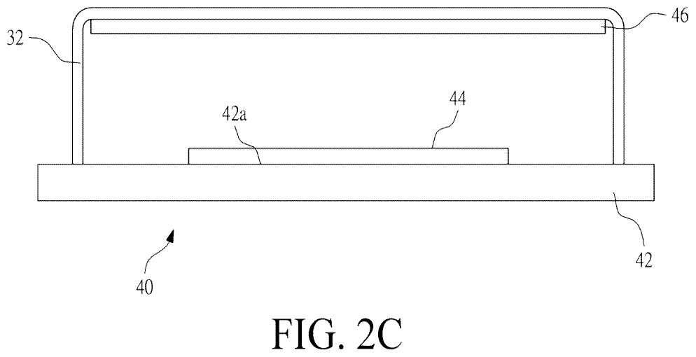

FIG. 2A to FIG. 2C are referred together, wherein FIG. 2A is a schematic view of a test component 20 provided by one embodiment of the disclosure; FIG. 2B is a schematic view of a test environment provided by one embodiment of the disclosure; and FIG. 2C is a schematic view of a test environment provided by another embodiment of the disclosure.

As shown in FIGS. 2A and 2B, in the case where a test substance can be cut, the test substance is cut into square slices as samples to be tested 22. Probiotics are coated on the surfaces to be tested 22a of the samples to be tested 22 and cover glasses 24 cover the surfaces to be tested 22a to complete the test components 20. In one embodiment, the samples to be tested are 5 cm×5 cm square slices and the cover glasses are 4 cm×4 cm square polyester film slice.

The test components 20 are placed in a culture dish 30. The culture dish 30 is covered with a glass cover 32. A glass pad 34 and a water-absorbing paper pad 36 are arranged under the test components 20 to ensure that the surfaces to be tested 22a are kept wet and that the test components 20 are stably placed in the space formed by the culture dish 30 and the glass cover 32. The culture dish 30 is placed in the constant-temperature environment (not shown) together with the test components 20 inside. In an embodiment, the constant-temperature environment is in an incubator.

Step S130 is referred together. In step S130, after the culture dish 30 is placed in the constant-temperature environment together with the test components 20 inside for the first time, and then the samples to be tested 22 are taken out and the surfaces to be tested 22a of the samples to be tested 22 are detected to generate the first detection values by using the adenosine triphosphate measuring instrument.

As shown in FIG. 2C, in the case that a test substance cannot be cut, the test substance 42 is directly used as a sample to be tested and the probiotics are directly coated on the flat surface (i.e. the surface to be tested 42a) of the test substance 42. A cover glass 44 covers the surface to be tested 42a to form a test component 40.

Unlike the test component 20 shown in FIG. 2B placed in the culture dish 30 to keep wet, the test component of the embodiment is oversized and cannot be placed in the culture dish 30. In the case, a glass cover 32 is covered on the surface to be tested 42a and is internally provided with a water-absorbing paper pad 46 so as to ensure that the surface to be tested 42a is kept wet. The test component 40 is placed in the constant-temperature environment continuously for the first time. After the first time, the surface to be tested 42a of the test substance 42 is detected to generate first detection values by using the adenosine triphosphate measuring instrument.

Then, as described in step S140, a plurality of first inhibition values is calculated by using the first detection values and the original values. In an embodiment, the first inhibition values are obtained by dividing differences between the original values and the first detection values by the original values.

Then, as described in step S150, the antimicrobial and antiviral effect of the material surface of the material to be tested is confirmed according to the first inhibition values. In an embodiment, if the first inhibition values are negative, it indicates that there is no antimicrobial and antiviral effect. If the first inhibition values are positive, that is, the original values are greater than the first detection values, it indicates that the antimicrobial and antiviral effect is achieved.

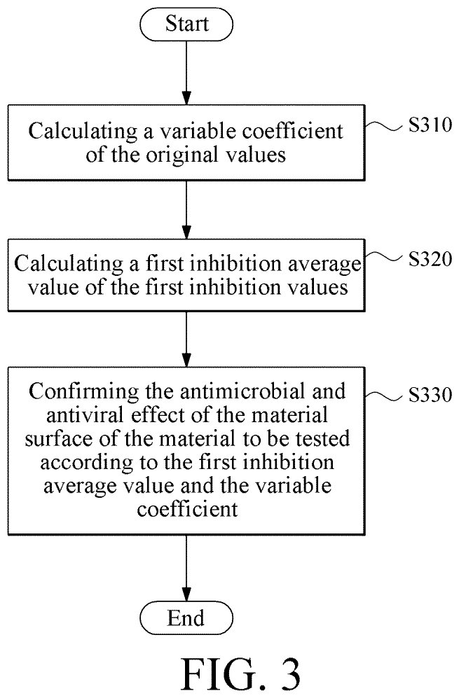

FIG. 3 shows an embodiment of step S150 in FIG. 1. Since a plurality of the samples to be tested are used for testing in the disclosure, in one embodiment, the antimicrobial and antiviral effect of the material surface of a material to be tested is confirmed by the following calculation steps in step S150.

First, as described in step S310, a variable coefficient (Cv) of the original values is calculated.

Then, as described in step S320, a first inhibition average value (D1) of the first inhibition values is calculated.

Then, as described in step S330, the antimicrobial and antiviral effect of the material surface of the material to be tested is confirmed according to the first inhibition average value and the variable coefficient. In an embodiment, if the first inhibition average value (D1) is negative and the variable coefficient (Cv) is smaller than a threshold, it indicates that the antimicrobial and antiviral effect cannot be determined. If the first inhibition average value (D1) is positive and the variable coefficient (Cv) is smaller than a threshold, it is determined that there is the antimicrobial and antiviral effect. In an embodiment, the threshold is 25%.

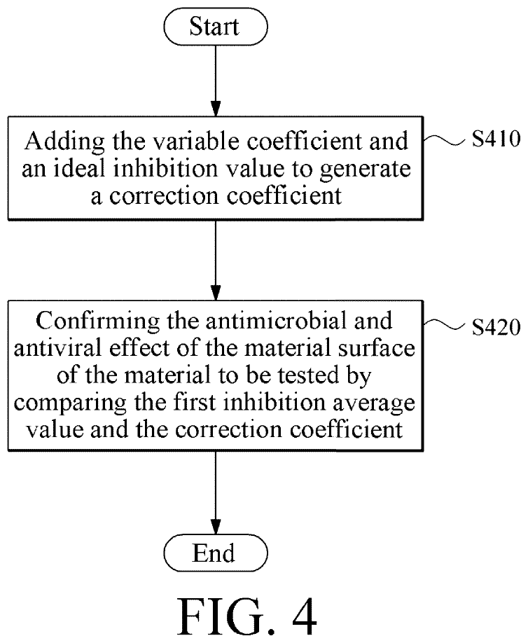

FIG. 4 shows an embodiment of step S330 in FIG. 3. Compared with the embodiment of FIG. 3, the embodiment includes a correction coefficient (QCv) to improve the determination accuracy.

First, as described in step S410, the variable coefficient (Cv) and an ideal inhibition value are added to generate the correction coefficient (QCv). In an embodiment, the ideal inhibition value is a preset value and 5%. The ideal inhibition value of 5% is approximately a hourly inhibition value converted from the antimicrobial and antiviral effect of 40% within ten hours.

Then, as described in step S420, the antimicrobial and antiviral effect of the material surface of the material to be tested is confirmed by comparing the first inhibition average value (D1) and the correction coefficient (QCv). In an embodiment, if the first inhibition average value (D1) is smaller than the correction coefficient (QCv), it indicates that the antimicrobial and antiviral effect cannot be determined. If the first inhibition average value (D1) is greater than the correction coefficient (QCv), it is determined that there is the antimicrobial and antiviral effect.

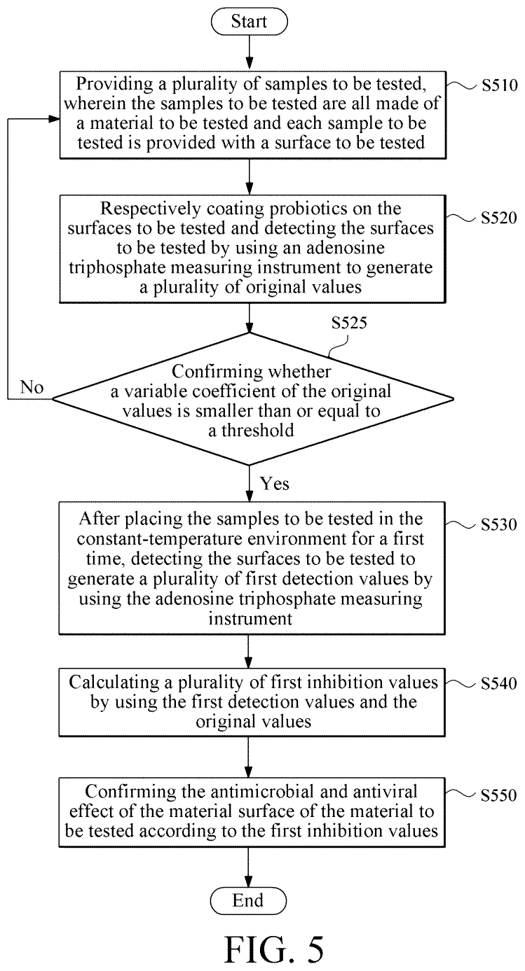

FIG. 5 is a flow diagram of a method for detecting an antimicrobial and antiviral effect provided by a second embodiment of the disclosure. Compared with the embodiment of FIG. 1, the embodiment further considers the situation that the differences between the samples to be tested are too large due to the defects of the preparation process when the plurality of samples to be tested are detected, which is specifically described as follows.

First, as described in step S510, a plurality of samples to be tested is provided, wherein the samples to be tested are all made of a material to be tested and each sample to be tested is provided with a surface to be tested.

Then, as described in step S520, probiotics are respectively coated on the surfaces to be tested and the surfaces to be tested are detected by using an adenosine triphosphate measuring instrument to generate a plurality of original values.

Then, as described in step S525, whether a variable coefficient of the original values is smaller than or equal to a threshold is confirmed. If the variable coefficient is smaller than or equal to the threshold, the process proceeds to step S530. If the variable coefficient of the original values is greater than the threshold, it indicates that the experiment fails, and the process returns to step S510 to start again. In an embodiment, the threshold is set to be 25%.

Then, as described in step S530, after the samples to be tested are placed in the constant-temperature environment for a first time, the surfaces to be tested are detected to generate a plurality of first detection values by using the adenosine triphosphate measuring instrument. Then, as described in step S540, a plurality of first inhibition values is calculated by using the first detection values and the original values. Then, as described in step S550, the antimicrobial and antiviral effect of the material surface of the material to be tested is confirmed according to the first inhibition values.

Steps S530 to S550 are similar to steps S130 to S150 of FIG. 1, which is not described herein.

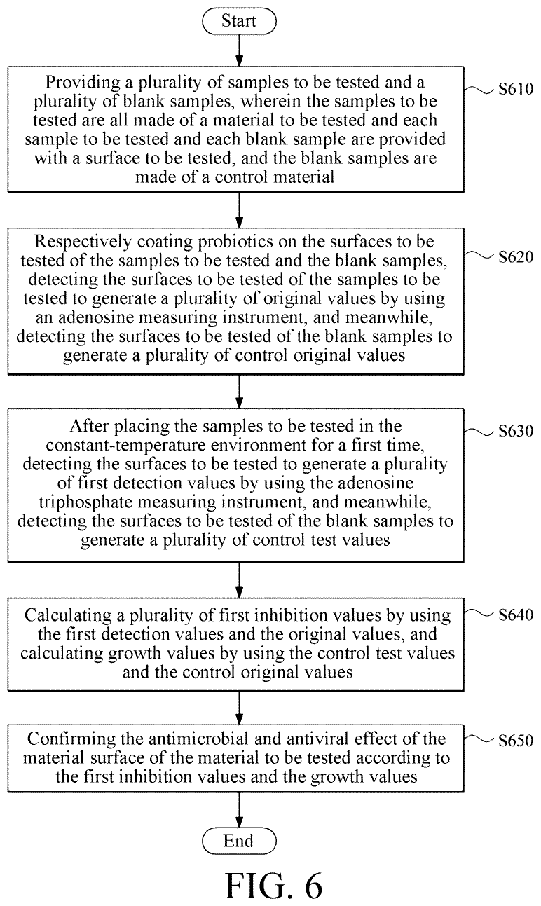

FIG. 6 is a flow diagram of a method for detecting an antimicrobial and antiviral effect provided by a third embodiment of the disclosure. Compared with the embodiment shown in FIG. 1, the embodiment compares the blank group with the test group so as to provide a more accurate detection result.

As shown in the figure, the method for detecting an antimicrobial and antiviral effect in the embodiment comprises the following steps.

First, as described in step S610, a plurality of samples to be tested and a plurality of blank samples are provided, wherein the samples to be tested are all made of a material to be tested and each sample to be tested and each blank sample are provided with the surface to be tested. The blank samples are made of a control material. In an embodiment, if it is determined whether a plastic material has an antimicrobial and antiviral effect by adding silver ions, a material without the addition of the silver ions is used as a control material and a material with the addition of the silver ions is used as a material to be tested. The blank samples form a blank group and the samples to be tested form a test group. In one embodiment, the number of the samples to be tested is three and the number of the blank samples is three.

Then, as described in step S620, probiotics are respectively coated on the surfaces to be tested of the samples to be tested and the blank samples, the surfaces to be tested of the samples to be tested are detected to generate a plurality of original values by using an adenosine measuring instrument, and meanwhile, the surfaces to be tested of the blank samples are detected to generate a plurality of control original values.

Then, as described in step S630, after the samples to be tested are placed in the constant-temperature environment for a first time, the surfaces to be tested of the samples to be tested are detected to generate a plurality of first detection values by using the adenosine triphosphate measuring instrument, and meanwhile, the surfaces to be tested of the blank samples are detected to generate a plurality of control test values.

It should be noted that the samples to be tested and the blank samples are prepared in the same manner to form the test components as shown in the second figure, and the test components are placed in the same constant-temperature environment for the first time.

Then, as described in step S640, a plurality of first inhibition values is calculated by using the first detection values and the original values, and growth values (G) are calculated by using the control test values and the control original values. In an embodiment, the first inhibition values are obtained by dividing differences between the original values and the first detection values by the original values. If the first inhibition values are positive, it indicates that there is an antimicrobial and antiviral effect. The growth value is an average value of values obtained by dividing differences between the control detection values and the control original values of the blank samples by the control original values. If the growth value is positive, it indicates that bacteria increase.

Then, as described in step S650, the antimicrobial and antiviral effect of the material surface of the material to be tested is confirmed according to the first inhibition values and the growth values. In an embodiment, if an average value of the first inhibition values is greater than an average value of the growth values, it is determined that there is the antimicrobial and antiviral effect.

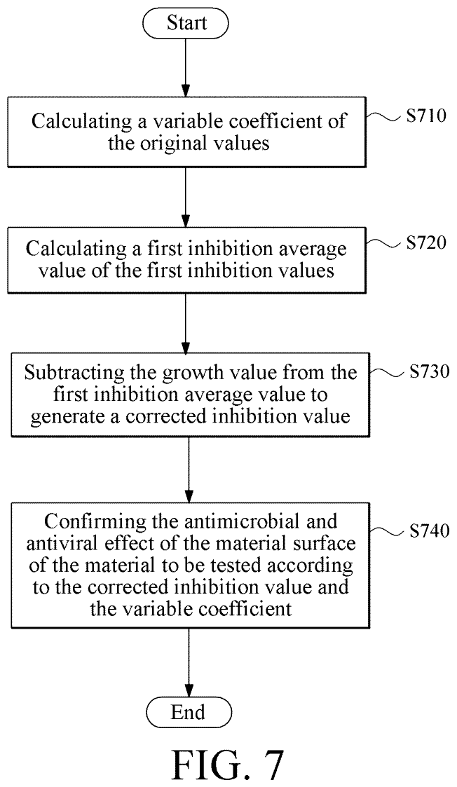

FIG. 7 shows an embodiment of step S650 in FIG. 6. Since a plurality of the samples to be tested are used for testing in the disclosure, in one embodiment, the antimicrobial and antiviral effect of the material surface of a material to be tested is confirmed by the following calculation steps in step S650.

First, as described in step S710, a variable coefficient (Cv) of the original values is calculated.

Then, as described in step S720, a first inhibition average value (D1) of the first inhibition values is calculated.

Then, as shown in step S730, the growth value (G) is subtracted from the first inhibition average value (D1) to generate a corrected inhibition value.

Then, as described in step S740, the antimicrobial and antiviral effect of the material surface of the material to be tested is confirmed according to the corrected inhibition value and the variable coefficient. In an embodiment, if the corrected inhibition value is negative and the variable coefficient (Cv) is smaller than a threshold, it indicates that the antimicrobial and antiviral effect cannot be determined. If the corrected inhibition value is positive and the variable coefficient (Cv) is smaller than a threshold, it is determined that there is the antimicrobial and antiviral effect. In an embodiment, the threshold is 25%.

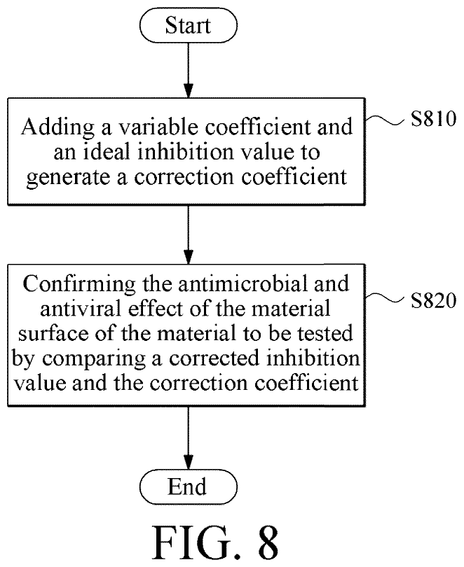

FIG. 8 shows an embodiment of step S740 in FIG. 7. Compared to the embodiment of FIG. 7, the embodiment includes a correction coefficient (QCv) to improve the determination accuracy.

First, as described in step S810, a variable coefficient (Cv) and an ideal inhibition value are added to generate the correction coefficient (QCv). In an embodiment, the ideal inhibition value is 5%. The ideal inhibition value of 5% is approximately a hourly inhibition value converted from the antimicrobial and antiviral effect of 40% within ten hours.

Then, as described in step S820, the antimicrobial and antiviral effect of the material surface of the material to be tested is confirmed by comparing a corrected inhibition value and the correction coefficient (QCv). In an embodiment, if the corrected inhibition value is smaller than the correction coefficient (QCv), it indicates that the antimicrobial and antiviral effect cannot be determined. If the corrected inhibition value is greater than the correction coefficient (QCv), it is determined that there is the antimicrobial and antiviral effect.

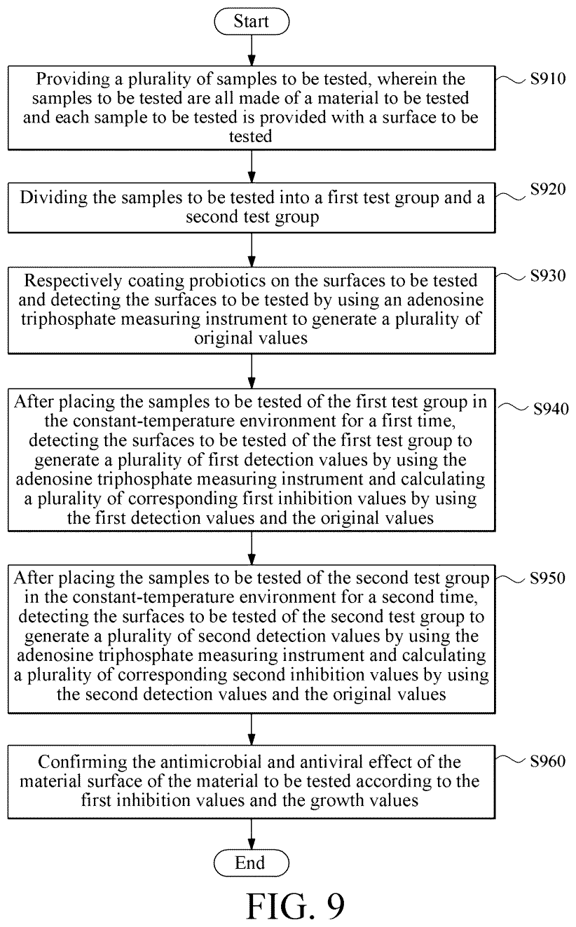

FIG. 9 is a flow diagram of a method for detecting an antimicrobial and antiviral effect provided by a fourth embodiment of the disclosure. Compared with the embodiment shown in FIG. 1, the embodiment divides the samples to be tested into a first test group and a second test group, and different placing times are set for the first test group and the second test group to determine the antimicrobial and antiviral effect of the material surface of the material to be tested. The method for detecting an antimicrobial and antiviral effect comprises the following steps.

First, as described in step S910, a plurality of samples to be tested is provided, wherein the samples to be tested are all made of a material to be tested and each sample to be tested is provided with a surface to be tested.

Then, as described in step S920, the samples to be tested are divided into a first test group and a second test group. In one embodiment, the number of the samples to be tested in the first test group is three and the number of the samples to be tested in the second test group is three.

Then, as described in step S930, probiotics are respectively coated on the surfaces to be tested and the surfaces to be tested are detected by using an adenosine triphosphate measuring instrument to generate a plurality of original values.

Then, as described in step S940, after the samples to be tested of the first test group are placed in the constant-temperature environment for a first time, the surfaces to be tested of the first test group are detected to generate a plurality of first detection values by using the adenosine triphosphate measuring instrument and a plurality of corresponding first inhibition values is calculated by using the first detection values and the original values. In an embodiment, the first inhibition values are obtained by dividing differences between the original values and the first detection values by the original values.

Then, as described in step S950, after the samples to be tested of the second test group are placed in the constant-temperature environment for a second time, the surfaces to be tested of the second test group are detected to generate a plurality of second detection values by using the adenosine triphosphate measuring instrument and a plurality of corresponding second inhibition values is calculated by using the second detection values and the original values. The second time is longer than the first time. In an embodiment, the second inhibition values are obtained by dividing differences between the original values and the second detection values by the original values. In one embodiment, the first time is one hour and the second time is two hours.

Then, as described in step S960, the antimicrobial and antiviral effect of the material surface of the material to be tested is confirmed according to the first inhibition values and the second inhibition values. In an embodiment, if an average value of the first inhibition values is smaller than an average value of the second inhibition values, it is determined that there is the antimicrobial and antiviral effect.

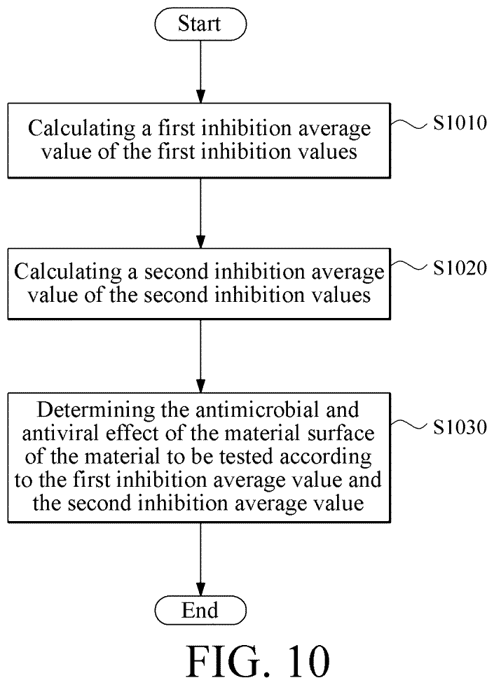

FIG. 10 shows an embodiment of step S960 in FIG. 9. Since a plurality of the samples to be tested are used for testing in the disclosure, in one embodiment, the antimicrobial and antiviral effect of the material surface of a material to be tested is confirmed by the following calculation steps in step S960. It should be noted that the embodiment is particularly suitable for confirming an antiviral effect of the material to be tested.

First, as described in step S1010, a first inhibition average value (D1) of the first inhibition values is calculated.

Then, as described in step S1020, a second inhibition average value (D2) of the second inhibition values is calculated.

Then, as described in step S1030, the antimicrobial and antiviral effect of the material surface of the material to be tested is determined according to the first inhibition average value (D1) and the second inhibition average value (D2). In an embodiment, if the first inhibition average value (D1) is smaller than the second inhibition average value (D2), it is determined that there is the antimicrobial and antiviral effect.

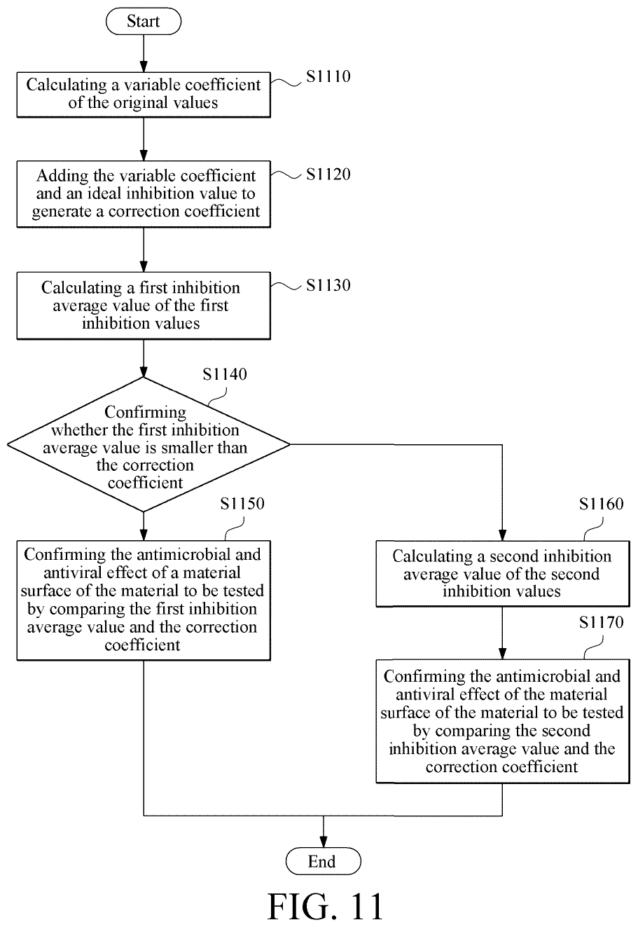

FIG. 11 shows another embodiment of step S960 in FIG. 9. Compared with the embodiment of FIG. 10, the embodiment includes a correction coefficient (QCv) to improve the determination accuracy. It should be noted that the embodiment is particularly suitable for confirming an antimicrobial effect of the material to be tested.

In one embodiment, the antimicrobial and antiviral effect of the material surface of a material to be tested is confirmed by the following calculation steps in step S960.

First, as described in step S1110, a variable coefficient (Cv) of the original values is calculated.

Then, as described in step S1120, the variable coefficient (Cv) and an ideal inhibition value are added to generate the correction coefficient (QCv). In one embodiment, the ideal inhibition value is 5%.

Then, as described in step S1130, a first inhibition average value (D1) of the first inhibition values is calculated.

Then, as described in step S1140, whether the first inhibition average value (D1) is smaller than the correction coefficient is confirmed.

If the first inhibition average value (D1) is greater than or equal to the correction coefficient (QCv), the embodiment directly uses the first inhibition values for determination. Then, the process proceeds to step S1150, the antimicrobial and antiviral effect of the material surface of the material to be tested is confirmed by comparing the first inhibition average value (D1) and the correction coefficient (QCv). In an embodiment, if the first inhibition average value (D1) is greater than the correction coefficient (QCv), it is determined that there is the antimicrobial and antiviral effect.

If the first inhibition average value (D1) is smaller than the correction coefficient (QCv), the embodiment further analyzes the second inhibition values. Then, the process proceeds to step S1160, a second inhibition average value (D2) of the second inhibition values is calculated. Then, as described in step S1170, the antimicrobial and antiviral effect of the material surface of the material to be tested is confirmed by comparing the second inhibition average value (D2) and the correction coefficient (QCv). In an embodiment, if the second inhibition average value (D2) is greater than the correction coefficient (QCv), it is determined that there is the antimicrobial and antiviral effect.

The method for detecting an antimicrobial and antiviral effect provided by the disclosure and a method for detecting an antiviral effect confirm the antimicrobial effect or the antiviral effectiveness of the test substance in a short time by using the adenosine triphosphate measuring instrument matched with a special detection process, and are helpful to reduce the detection cost and time spent.

Although the disclosure is provided as the foregoing in the manner of embodiments, however, which are not used to limit the disclosure, any person skilled in the art can make various changes and modification without departing from the spirits and scope of the disclosure. Therefore, the protection scope of the disclosure is determined by the attached claims.

What is claimed is:

1. A method for detecting an antimicrobial and antiviral effect, suitable for detecting the antimicrobial and antiviral effect of a material surface of a material to be tested, and the method comprising:
providing a plurality of samples to be tested, wherein the samples to be tested are all made of the material to be tested and each sample to be tested is provided with a surface to be tested;
respectively coating probiotics on the surfaces to be tested and detecting the surfaces to be tested by using an adenosine triphosphate (ATP) measuring instrument to generate a plurality of original values;
after placing the samples to be tested in the constant-temperature environment for a first time, detecting the surfaces to be tested to generate a plurality of first detection values by using the adenosine triphosphate measuring instrument;
calculating a plurality of first inhibition values by using the first detection values and the original values; and
confirming the antimicrobial and antiviral effect of the material surface of the material to be tested according to the first inhibition values.

2. The method for detecting an antimicrobial and antiviral effect according to claim 1, after the step of detecting the surfaces to be tested by using the adenosine triphosphate measuring instrument to generate the original values, further comprising:
covering the surfaces to be tested with a plurality of cover glasses respectively to form a plurality of test components; and
placing the test components in a culture dish to remain wet and placing the culture dish in the constant-temperature environment.

3. The method for detecting an antimicrobial and antiviral effect according to claim 1, wherein the number of the samples to be tested is three.

4. The method for detecting an antimicrobial and antiviral effect according to claim 1, after the step of detecting the surfaces to be tested by using the adenosine triphosphate measuring instrument to generate the original values, further comprising:
confirming whether a variable coefficient of the original values is less than or equal to a threshold value.

5. The method for detecting an antimicrobial and antiviral effect according to claim 1, further comprising:
providing a blank sample, wherein the blank sample is provided with a control surface to be tested;
coating the probiotics on the control surface to be tested and detecting the control surface to be tested by using the adenosine triphosphate measuring instrument to generate a control original value;
after placing the blank sample in the constant-temperature environment for the first time, detecting the control surface to be tested to generate a control test value by using the adenosine triphosphate measuring instrument and
calculating a growth value by using the control original value and the control test value.

6. The method for detecting an antimicrobial and antiviral effect according to claim 5, wherein the step of confirming the antimicrobial and antiviral effect of the material surface of the material to be tested according to the first inhibition values comprises:
calculating a variable coefficient of the original values;
calculating a first inhibition average value of the first inhibition values;
subtracting the growth value from the first inhibition average value to generate a corrected inhibition value; and
confirming the antimicrobial and antiviral effect of the material surface of the material to be tested according to the corrected inhibition value and the variable coefficient.

7. The method for detecting an antimicrobial and antiviral effect according to claim 6, wherein the step of confirming the antimicrobial and antiviral effect of the material surface of the material to be tested according to the corrected inhibition value and the variable coefficient comprises:
adding the variable coefficient and an ideal inhibition value to generate a correction coefficient; and
confirming the antimicrobial and antiviral effect of the material surface of the material to be tested by comparing the corrected inhibition value and the correction coefficient.

8. The method for detecting an antimicrobial and antiviral effect according to claim 1, wherein the step of confirming the antimicrobial and antiviral effect of the material surface of the material to be tested according to the first inhibition values comprises:
calculating a variable coefficient of the original values;
calculating a first inhibition average value of the first inhibition values; and
confirming the antimicrobial and antiviral effect of the material surface of the material to be tested according to the first inhibition average value and the variable coefficient.

9. The method for detecting an antimicrobial and antiviral effect according to claim 8, wherein the step of confirming the antimicrobial and antiviral effect of the material surface of the material to be tested according to the first inhibition average value and the variable coefficient comprises:
adding the variable coefficient and an ideal inhibition value to generate a correction coefficient; and
confirming the antimicrobial and antiviral effect of the material surface of the material to be tested by comparing the first inhibition average value and the correction coefficient.

10. The method for detecting an antimicrobial and antiviral effect according to claim 1, wherein the probiotics is Bacillus subtilis.

11. A method for detecting an antimicrobial and antiviral effect, suitable for detecting the antimicrobial and antiviral effect of a material surface of a material to be tested, and the method comprising:
providing a plurality of samples to be tested, wherein the samples to be tested are all made of the material to be tested and each sample to be tested is provided with a surface to be tested;
dividing the samples to be tested into a first test group and a second test group;
respectively coating probiotics on the surfaces to be tested and detecting the surfaces to be tested by using an adenosine triphosphate measuring instrument to generate a plurality of original values;
after placing the samples to be tested of the first test group in the constant-temperature environment for a first time, detecting the surfaces to be tested of the first test group to generate a plurality of first detection values by using the adenosine triphosphate measuring instrument and calculating a plurality of corresponding first inhibition values by using the first detection values and the original values;
after placing the samples to be tested of the second test group in the constant-temperature environment for a second time, detecting the surfaces to be tested of the second test group to generate a plurality of second detection values by using the adenosine triphosphate measuring instrument and calculating a plurality of corresponding second inhibition values by using the second detection values and the original values; and
confirming the antimicrobial and antiviral effect of the material surface of the material to be tested according to the first inhibition values and the second inhibition values.

12. The method for detecting an antimicrobial and antiviral effect according to claim 11, wherein the second time is longer that the first time.

13. The method for detecting an antimicrobial and antiviral effect according to claim 12, wherein the first time is one hour and the second time is two hours.

14. The method for detecting an antimicrobial and antiviral effect according to claim 11, wherein the step of confirming the antimicrobial and antiviral effect of the material surface of the material to be tested according to the first inhibition values and the second inhibition values comprises:
calculating a first inhibition average value of the first inhibition values;
calculating a second inhibition average value of the second inhibition values; and
confirming the antimicrobial and antiviral effect of the material surface of the material to be tested according to the first inhibition average value and the second inhibition average value.

15. The method for detecting an antimicrobial and antiviral effect according to claim 11, wherein the step of confirming the antimicrobial and antiviral effect of the material surface of the material to be tested according to the first inhibition values and the second inhibition values comprises:
calculating a variable coefficient of the original values;
adding the variable coefficient and an ideal inhibition value to generate a correction coefficient;
calculating a first inhibition average value of the first inhibition values and confirming the antimicrobial and antiviral effect of the material surface of the material to be tested by comparing the first inhibition average value and the correction coefficient; and
when the first inhibition average value is less than the correction coefficient, calculating a second inhibition average value of the second inhibition values and confirming the antimicrobial and antiviral effect of the material surface of the material to be tested by comparing the second inhibition average value and the correction coefficient.
